(19) United States
(12) Patent Application Publication
(10) Pub. No.: US 2013/0210094 A1
(43) Pub. Date: Aug. 15, 2013

(54) HETEROGENEOUS ENZYMATIC CATALYST, PROCESS FOR PREPARING SAME AND USE FOR CONTINUOUS FLOW ENZYMATIC CATALYSIS

(71) Applicants: Renal Backov, Bordeaux-Cauderan (FR); Clement Sanchez, Bure Sur Yvette (FR); Nicolas Brun, Bordeaux (FR); Herve Deleuze, Saint Magne (FR)

(72) Inventors: Renal Backov, Bordeaux-Cauderan (FR); Clement Sanchez, Bure Sur Yvette (FR); Nicolas Brun, Bordeaux (FR); Herve Deleuze, Saint Magne (FR)

(73) Assignees: CENTRE NATIONAL DE LA RECHERCHE SCIENTIFIQUE, PARIS (FR); UNIVERSITE PIERRE ET MARIE CURIE (PARIS 6), PARIS (FR)

(21) Appl. No.: 13/808,766

(22) PCT Filed: Jul. 25, 2011
(86) PCT No.: PCT/FR2011/051785

§ 371 Date: Apr. 26, 2013

(30) Foreign Application Priority Data
Jul. 26, 2010 (FR) 1056099

(51) Int. Cl. C12N 11/14; C12P 7/62; C12P 7/64

(52) U.S. Cl. CPC C12N 11/14; C12P 7/6418; C12P 7/62; C12P 7/649
USPC 435/134; 435/176; 435/135

(57) ABSTRACT

The present invention relates to a heterogeneous enzymatic catalyst consisting of a macroprous silica monolith incorporating an enzyme immobilized by means of a compiling agent, to a process for preparing this enzymatic catalyst, to the use of the catalyst for carrying out chemical reactions by continuous flow heterogeneous enzymatic catalyst and to a process of continuous flow heterogeneous enzymatic catalysis using said catalyst.

The present invention relates to a heterogeneous enzymatic catalyst consisting of a macroporeuse silica monolith incorporating an enzyme immobilized by means of a coupling agent, to a process for preparing this enzymatic catalyst, to the use of this catalyst for carrying out chemical reactions by continuous flow heterogeneous enzymatic catalysis and to a process of continuous flow heterogeneous enzymatic catalysis using said catalyst.

Given that oil-derived fuels are non-renewable energy sources and that their use generates an accumulation of carbon dioxide, of pollutants and of potentially carcinogenic compounds in the environment, considerable efforts have been made to develop alternative “green” fuels, i.e. fuels which are renewable, biodegradable and nontoxic. In this context, biodiesel (methyl or ethyl esters of fatty acids), a natural fuel obtained from plants or from waste plant oils, has aroused considerable attention over the past ten years. Biodiesel is obtained by transesterification of plant oil triglycerides with an alcohol, such as methanol or ethanol. The oil triglycerides are esters of glycerol (also referred to as glycerin) and of fatty acids R-COOH.

By way of example, the reaction for transesterification of oil triglycerides with methanol can be represented by the following reaction scheme:

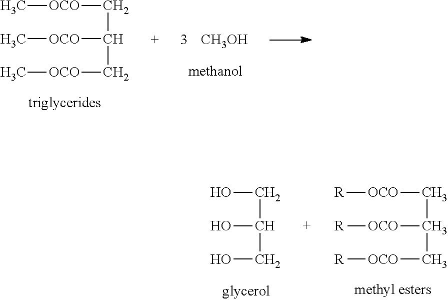

There are two major types of biodiesel production process: homogeneous-phase catalysis processes, using catalysts that are soluble in the reaction medium, and heterogeneous-phase catalysis processes, using catalysts which are not soluble in the reaction medium.

At the current time, biodiesel production is mainly carried out by homogeneous-phase catalysis. It consists in carrying out the transesterification of the triglycerides in the presence of acid catalysts, such as inorganic acids (HCl, H₂SO₄) or sulfonic acids, or else basic catalysts, such as hydroxides, alkoxides, alkali metal or alkaline-earth metal soaps, or alternatively amines of the guanidine family, for example. A greater reactivity is generally obtained in a basic medium. The acid catalysts are used less often because of their lower reactivity (they are approximately 4000 times slower than basic catalysts) and the high risks of corrosion of industrial equipment, These processes can be implemented in discontinuous flows or in continuous flows. Although processes for biodiesel production by alkaline homogeneous-phase catalysis are inexpensive to implement and highly reactive, they have, however, the major drawback of being highly energy consuming. Furthermore, the presence both of water and of fatty acids in the reaction medium generates a partial saponification reaction which leads to a loss of catalytic efficiency (H. Fukuda et al, Journal of Bioscience and Bioengineering, 2001, 92, 405-416).

Recently, enzymatic transesterification processes using a lipase have been presented as an advantageous alternative for the synthesis of biodiesel because these processes make it possible to do away with the drawbacks encountered with the homogeneous catalysis process (M. S. Antczak et al, Renewable Energy, 2009, 34, 1185-1194). Lipases make it possible to catalyze the hydrolysis of plant oils specifically and under mild conditions, firstly, while releasing glycerol and, secondly, in the presence of a short-chain alcohol, to promote the formation of linear-chain esters. Furthermore, at the end of the reaction, it is easy to recover the glycerol, which is the by-product of the transesterification reaction, and the purification of the fatty acid esters is simple to carry out.

The main obstacle to the industrialization of these processes is the cost of producing the enzymes, and also the rigorous control of the reaction parameters during the transesterification reaction.

One of the solutions to this problem consists in immobilizing the enzymes on a solid support. The term heterogeneous enzymatic catalysis is then used. Heterogeneous enzymatic catalysis consists in carrying out plant oil triglyceride transesterification in the presence of a catalyst which is insoluble in the reaction medium. Heterogeneous enzymatic catalysis has significant advantages in terms of being environmentally friendly. In particular, it meets the criteria associated with the new concepts of “green chemistry”, since the purity of the products obtained, associated with high synthesis yields, results in a virtually total disappearance of polluting discharges. Furthermore, the absence of salts in the reaction products does not impose, unlike homogeneous-phase catalysis, expensive purification treatments, and broadens the possibilities of industrial outlets.

A process for conversion of cooking oils into biodiesel using a Rhizopus oryzae lipase immobilized on a solid support has, for example, already been proposed, in particular by Chen G. et al, Biodiesel. Appl., Biochem. Biotech., 2006, 132 (1-3), 911-921, The authors indicate that, under the most optimal conditions, the conversion rate to methyl esters is 88-90%. The nature of the solid support on which the lipase is immobilized is not indicated in this article. More recently, Li et al., Process Biochem., 2009, 44 (6), 685-688, have proposed a process for enzymatic conversion of cooking oils into biodiesel in an organic solvent medium using a Penicillium expansum lipase adsorbed in a silica gel. However, the authors indicate that the presence of water derived from the esterification reaction is detrimental to obtaining a high level of methyl esters.

The various enzymatic catalytic systems currently proposed consist of a generally porous solid support (polymer matrices, chlorosilica, calcium silicate, zeolites, zirconium, kaolinite, porous glass, alumina, etc.), on which an enzyme is immobilized. The solid supports of the catalysts used in heterogeneous catalysis are in particular acidic zeolites, heteropolyacids, ion exchange resins and sulfonic acids immobilized on solid supports, sulfate zirconias and mixed metal oxides. Most commonly, the solid catalysts in which the solid support is a zeolite are in powder form and must be dispersed in the reaction medium in which they must be present in order to catalyze a reaction. Given the small size of the particles, the recovery of these catalysts from the reaction medium is a restrictive step. The other solid supports that can be used in heterogeneous catalysis are not, moreover, entirely satisfactory from the point of view of their mechanical strength and their temperature resistance.

In order to achieve high production levels, in particular of biodiesel, it is important to be able to make these heterogeneous catalysts operate in continuous flow.

As it happens, not all the solid supports on which it is possible to immobilize the enzymes are, however, compatible with a continuous flow heterogeneous catalysis process. Moreover, the supports that can be used in continuous flow do not always make it possible to achieve high conversion rates which are stable over time. Indeed, the first indispensible condition is to have a monolithic material. The second condition is that this material has an interconnected macroporosity so as to allow the reaction medium to flow. The third condition is that the mechanical properties of the monolith withstand the flow imposed and the temperature over a long period of time.

There is therefore a need for a heterogeneous enzymatic catalyst:
which can be used in a continuous flow catalysis process (not requiring an intermediate regeneration step), in particular for catalyzing the production of biodiesel by transesterification of the fatty acid triglycerides present in plant oils;
which can be used over a long period of time without significant loss of the catalytic activity;
which can be prepared according to a process that is simple to implement,
which allows the use of unpurified enzymes with a very high catalytic efficiency.

This objective is achieved with the heterogenerous enzymatic catalyst which is the subject of the present invention and which will be described hereinafter.

The subject of the present invention is a heterogeneous enzymatic catalyst, characterized in that it is in the form of a cellular monolith consisting of a silica matrix, said monolith being free of micropores and comprising macropores having a mean size d_{A} of from 1 μm to 100 μm and mesopores having a mean size d_{E} of from 2 to 50 nm, said macropores being interconnected, and in which the internal surface of the macropores is functionalized with a coupling agent, chosen from the silanes, to which an enzyme is attached, by means of a covalent or electrostatic bond.

According to one preferred embodiment of the invention, the immobilized enzyme is an unpurified enzyme. Indeed, as is demonstrated in the examples illustrating the present application, the use of such a monolith makes it possible to employ an unpurified enzyme for the continuous flow catalysis of a chemical reaction, and quite particularly, when the enzyme is a lipase, of a fatty acid triglyceride transesterification reaction.

For the purpose of the present invention, the term “unpurified enzyme” is intended to mean any protein material comprising at least one nonisolated enzyme that has undergone no purification step.

The term “monolith” is intended to mean a solid object having a mean size of at least 1 mm.

According to one preferred embodiment of the invention, the mean size d_{A} of the macropores ranges from 10 to 100 μm, and even more preferentially approximately from 20 to 70 μm. The process for preparing the monoliths in accordance with the invention has the advantage of not requiring a sintering step that would lead to a shrinkage of the size of the macropores. Thus, the preparation process in accordance with the invention and that will be described hereinafter makes it possible to obtain monoliths in which the macropores preferably have the sizes indicated above, the latter being particularly suitable for carrying out continuous flow enzymatic catalysis.

In this monolith, the walls of the macropores generally have a thickness of from 0.5 to 40 μm, and preferably from 2 to 25 μm.

The specific surface area of the monolith is generally approximately from 200 to 1000 m²/g, preferentially approximately from 100 to 300 m²/g.

According to the invention, the bond which attaches the coupling agent to the silica is an iono-covalent bond.

According to one preferred embodiment of the invention, the coupling agent is chosen from silanes chosen from the group consisting of γ-glycidoxypropyltrimethoxysilane; silylated ionic liquids, such as, for example, 1-methyl-3-(3-triethoxysilylpropyl)imidazolium chloride or 1-methyl-3-(3-triethoxysilylpropyl)imidazolium hexafluorophosphate; silanes of formula Si(OR²)₃R³ in which R² represents a C₁-C₂ alkyl group, and R³ represents a —(CH₂OH—CH₂OH)_{q}—CH₂OH or —(CH₂OH—CH₂OH)_{q}—CH₂CH₃ group in which q is an integer ranging from 1 to 10.

Among such silanes, γ-glycidoxypropyltrimethoxysilane, also known as “Glymo”, is particularly preferred.

The nature of the enzyme that can be immobilized on the silica monolith by means of the coupling agent is not critical provided that it comprises at least one functional group capable of reacting with a complementary functional group borne by the coupling agent so as to form an iono-covalent bond. When the coupling agent used is a silylated ionic liquid, electrostatic bonds are involved.

According to one preferred embodiment of the invention, the enzyme is chosen from:

i) hydrolases (class EC 3 of the classification established by the Enzyme Commission, Brussels), such as esterases (EC 3.1), and in particular carboxylic ester hydrolases (EC 3.1.1) such as lipases (EC 3.1.1.3 or triacylglycerol acylhydrolases); aminoacylases (EC 3.5.1.14), amidases (EC 3.5.1.4; EC 3-5-1-3 or ω-amidase; EC 3-5-1-11 or penicillin amidase); nitrilases (class EC 3.5.5.1.) which catalyze the hydrolysis of nitriles to carboxylic acids;

ii) lyases (class EC 4) comprising in particular carboxy-lyases (EC 4.1.1), aldehyde-lyases (EC 4.1.2.) such as oxynitrilases (classes EC 4-1-2-10 and EC 4-1-2-37) catalyzing the synthesis of chiral cyanohydrins; and hydro-lyases (EC 4.2.1);

iii) isomerases (EC 5) comprising in particular epimerases and racemases (EC 5.1.), in particular epimerases and racemases of class EC 5.1.1. that catalyze the formation of enantiomers of amino acids; and

iv) oxidoreductases (EC 1) comprising in particular glucose oxidases (EC 1.1.3.4) such as Aspergillus niger glucose oxidase and peroxidases (EC 1.11.1) such as horseradish peroxidase.

According to one particularly preferred embodiment of the invention, the heterogeneous catalyst is intended to be used in a process for producing biodiesel by fatty acid triglyceride transesterification and the enzyme is chosen from lipases of microbial or plant origin, and in particular from Candida rugosa, Candida antartica, Aspergillus niger, Aspergillus oryzae, Thermomyces lanuginosus, Chromobacterium viscosum, Rhizomucor miehei, Pseudomonas fluorescens, Pseudomonas cepacia, Penicillium roqueforti, Penicillium expansum and Rhizopus arrhizus lipases and wheatgerm lipases.

The amount of enzymes immobilized within the catalyst in accordance with the invention may be determined by thermogravimetric analysis and by elemental analysis. According to one preferred embodiment of the invention, the amount of enzyme immobilized ranges from 1% to 40% by weight approximately and more preferentially from 3% to 20% by weight approximately, relative to the total weight of the catalyst.

A subject of the present invention is also a process for preparing a heterogeneous enzymatic catalyst in accordance with the invention and as defined above, said process comprising the following steps:

1) a first step of preparing a cellular monolith consisting of a silica matrix, said monolith being free of micropores and comprising macropores having a mean size d_{A} of from 1 μm to 100 μm and mesopores having a mean size d_{E} of from 2 to 50 nm, said pores being interconnected, said first step comprising the following substeps:
1a) preparing an emulsion by introducing an oily phase into an aqueous surfactant solution,
1b) adding an aqueous solution of at least one silica oxide precursor to the surfactant solution, before or after preparation of the emulsion,
1c) introducing the reaction mixture into a mold,
1d) leaving the reaction mixture to stand in the mold until said silica precursor has condensed in the shape of said monolith,
2d) washing said monolith, in continuous flow, with an organic solvent;

2) a second step of functionalizing the internal surface of the macropores with a coupling agent chosen from silanes, by impregnating the cellular monolith, in continuous flow, with a solution of the coupling agent in an organic solvent; and

3) a third step of immobilizing at least one enzyme on the coupling agent by means of a covalent bond, by impregnating the thus functionalized monolith, in continuous flow, with an aqueous solution or an aqueous dispersion of at least one enzyme.

According to one particular and preferred form of the invention, the mold used during the first step is itself contained inside a device allowing continuous flow circulation of a liquid, such as, for example, a chromatography column.

According to the process in accordance with the invention, no step of sintering the silica matrix is required, which makes it possible to obtain monoliths in which the size of the macropores ranges preferentially from 10 to 100 μm, and even more preferentially from 20 to 70 μm.

According to one preferred embodiment of the invention, the continuous flows mentioned in steps 2) and 3) of said process are ascending continuous flows so as to optimize the distribution of the coupling agent, and then of the enzyme, in the whole of the volume of the macropores of the cellular silica monolith.

During steps 2) and 3), the flow rate ranges preferably from 0.02 to 0.1 ml/min.

The functionalizing step 2) is preferably carried out at ambient temperature, and for a period of 24 hours and even more preferentially for a period of approximately 72 hours.

The immobilizing step 3) is preferably carried out at ambient temperature, and for a period of 72 hours and even more preferentially for a period of approximately 120 hours (5 days). Optionally, this step can be repeated twice. In this case, the process also comprises, before carrying out step 3) for the second time, an additional step of impregnating the monolith, in continuous flow, with a solution of an aldehyde, such as, for example, glutaraldehyde. This step brings about the attachment of the aldehyde to the amino groups of the enzyme previously attached to the coupling agent and allows the subsequent immobilization of a second layer of enzymes.

According to the invention, the silica precursor(s) is (are) chosen from tetramethoxyorthosilane (TMOS), tetraethoxyorthosilane (TEOS), dimemthyldiethoxysilane (DMDES), mixtures of DMDES with TEOS or TMOS, mixtures of TMOS or of TEOS with γ-glycidoxypropyltrimethoxysilane, and mixtures of DMDES or of γ-glycidoxypropyltrimethoxysilane with a silicate.

According to one preferred embodiment of the invention, the silica precursor is TEOS.

The concentration of silica oxide precursor(s) within the aqueous solution is preferably greater than 10% by weight relative to the weight of the aqueous phase. This concentration ranges more preferentially from 17% to 35% by weight relative to the weight of the aqueous phase.

The oily phase of the emulsion prepared in step 1) is preferably made up of one or more compounds chosen from linear or branched alkanes having at least 12 carbon atoms. By way of example, mention may be made of dodecane and hexadecane. The oily phase can also be made up of a silicone oil of low viscosity, i.e. less than 400 centipoises.

The amount of oily phase present within the emulsion can be adjusted according to the diameter of the macropores that it is desired to obtain for the silica matrix, it being understood that, the higher the oil/water volume fraction, the smaller the diameter of the oil droplets within the emulsion and also the smaller the diameter of the macropores.

In general, the oily phase represents from 60% to 90% by volume relative to the total volume of the emulsion. This amount of oil makes it possible to obtain a silica matrix in which the mean diameter of the macropores ranges from 1 to 100 μm approximately.

The surfactant compound may be a cationic surfactant chosen in particular from tetradecyltrimethylammonium bromide (TTAB), dodecyltrimethylammonium bromide or cetyltrimethylammonium bromide. When the surfactant compound is cationic, the reaction medium is brought to a pH of less than 3, preferably less than 1. Tetradecyltrimethylammonium bromide is particularly preferred.

Finally, the surfactant compound may be a nonionic surfactant chosen from surfactants with an ethoxylated head group and nonylphenols. Among such surfactants, mention may in particular be made of block copolymers of ethylene glycol and of propylene glycol, sold, for example, under the trade names Pluronic® P123 and Pluronic® F127 by the company BASF. When the surfactant compound is nonionic, the reaction medium is brought to a pH of greater than 10 or less than 3, preferably less than 1, and also preferably contains sodium fluoride in order to improve the condensation of the silica oxide precursors.

The total amount of surfactant present within the emulsion may also be adjusted according to the diameter of the macropores that it is desired to obtain in the silica template. This amount can also vary according to the nature of the surfactant used.

In general, the amount of surfactant ranges from 1% to 10% by weight, preferably from 3% to 6% by weight, relative to the total weight of the emulsion.

The step of condensing the silica oxide precursor(s) is advantageously carried out at a temperature close to ambient temperature. The duration of this step may vary from a few hours (2 to 3 hours to a few weeks (2 to 3 weeks) depending on the pH of the reaction medium.

According to one preferred embodiment of the invention, the organic solvent used for washing the silica matrix obtained at the end of the first step is chosen from tetrahydrofuran, and acetone, and mixtures thereof.

The solvent of the coupling agent solution used during the functionalizing step 2) is an organic solvent, preferably chosen from chloroform and toluene, and mixtures thereof. Said solvent is preferentially chloroform.

The amount of coupling agent in the solution used for the functionalizing step can be adjusted according to the diameter of the macropores of the silica monolith and the amount of enzyme that it is desired to immobilize. In general, this amount can range from 0.02 M to 0.5 M, and preferably from 0.05 M to 0.2 M.

According to one particular and preferred embodiment of the invention, a solution of coupling agent at 0.05 M in chloroform is used.

According to one preferred embodiment of the process in accordance with the invention, the monolith functionalized with the coupling agent, as obtained at the end of the functionalizing step 2), is washed, under continuous flow, with an organic solvent, such as, for example, tetrahydrofuran, chloroform or acetone, and then subsequently with distilled water.

Also preferably, at the end of the immobilizing step 3, the monolith is preferably washed, in continuous flow, with distilled water.

The heterogeneous enzymatic catalyst in accordance with the present invention can be used for carrying out continuous flow heterogeneous-phase catalyzed chemical reactions. The nature of the chemical reactions capable of being catalyzed by the catalyst in accordance with the invention will of course vary depending on the nature of the unpurified enzyme which is immobilized.

Thus, when the unpurified enzyme is a lipase, the catalyst in accordance with the invention is used for catalyzing the hydrolysis of fatty acid triglycerides, esterification reactions between an acid and an alcohol, transesterification reactions between an ester and an alcohol, inter-esterification reactions between two esters or reactions for transfer of an acetyl group of an ester to an amine or to a thiol.

In particular, when the enzyme is a lipase, said catalyst can be used, for example, for catalyzing:
the synthesis of butyl oleate, which is a lubricant for biodiesels;
the hydrolysis of glycerol-linoleic ester derivatives to result in soaps or detergents;
reactions for transesterification of fatty acid triglycerides with an alcohol, said reactions being involved in the synthesis of low-viscosity biodiesels.

Finally, a subject of the present invention is a process of heterogeneous enzymatic catalysis using said catalyst. This process is characterized in that it is carried out by passing a liquid reaction medium in ascending continuous flow through said heterogeneous catalyst.

The flow rate can vary according to the nature of the enzyme immobilized in the catalyst. In general, the flow rate ranges from 0.02 to 0.2 ml/min.

According to one particular and preferred embodiment of the invention, the heterogeneous catalysis process is a biodiesel production process, therefore the reaction medium comprises fatty acid triglycerides and the enzyme incorporated into the heterogeneous catalyst is a lipase.

The present invention is illustrated by the following exemplary embodiments, to which the invention is not, however, limited.

### EXAMPLES

The raw materials used in the examples which follow are listed below:
98% tetradecyltrimethylammonium bromide (TTAB): from Fluka;
98% tetraethoxyorthosilane (TEOS): from Fluka;
99% dodecane: from Fluka;
Coupling agent: γ-glycidoxypropyltrimethoxysilane sold under the trade name Glymo by Sigma Aldrich (St-Louis, Mo.);
Candida rugosa lipase, EC 3.1.1.3, type VII, at 700 U/mg, from Sigma Chemicals (St Louis, Mo.);
Thermomyces lanuginosus lipase, solution at at least 100 000 U/g, from Sigma Aldrich (St. Louis, Mo.);
oleic acid, glyceryl trilinoleate (98%), ethyl linoleate (≧98%), linoleic acid (≧99%), n-heptane, ethanol, 1-butanol, from Sigma Aldrich (Paris, France).

The other chemical products and solvents used in the examples were all of analytical grade or HPLC grade.

These raw materials were used as received from the manufacturers, without additional purification.

Characterizations:

The macroporosity was characterized qualitatively by means of a scanning electron microscopy (SEM) technique using a scanning electron microscope sold under the reference JSM-840A by the company JEOL, operating at 10 kV. The samples were coated with gold or carbon before they were characterized.

The macroporosity was quantified by mercury intrusion measurements using an instrument sold under the name Micromeritics Autopore IV, in order to obtain the characteristics of the macroscopic cells making up the monolith backbone.

The specific surface area measurements and the mesoscopic-scale characterizations were made by means of nitrogen adsorption-desorption techniques using an instrument sold under the name Micromeritics ASAP 2010; the analysis being carried out by BET or BJH calculation methods.

The mesoporosity was characterized qualitatively by means of a transmission electron microscopy (TEM) technique using a microscope sold under the reference H7650 by the company Hitachi, having an accelerating voltage of 80 kV, and coupled to a camera sold under the reference Orius 11 MPX by the company Gatan Inc.

Analyses by high performance liquid chromatography (HPLC) were carried out on a system equipped with manual injection 600 solvent pumps (Waters, Milford, Mass., USA), in an isocratic system, and using acetonitrile as mobile phase. The compounds were separated on an Atlantis dC18 chromatography column (4.6 mm×150 mm, 5 μm) with an Atlantis dC18 guard column (Waters). The columns were used at ambient temperature. The Empower® software (Waters) was used for data acquisition and processing, The standards were dissolved in methyl t-butyl ether (MTBE). All the solutions were filtered through a 0.45 μm membrane and degassed before use. The flow rate of the liquid phase was set at 1 ml/min and the volume of the samples injected was 20 μl. The catalyzed esterification reactions were monitored using a refractometer sold under the reference 410 by the company Waters (Milford, Mass., USA), For the detection of the products resulting from the catalyzed hydrolysis and transesterification reactions, the system was equipped with an ultraviolet (UV) diode-array detector (WAT996, Waters, Milford, Mass., USA). The measurements were carried out at a wavelength of 204 nm, which corresponded to the maximum absorbance. The following elution gradient was used: (solvent A: acetonitrile, solvent B: MTBE): A/B: 100/0 (v/v) isocratic for 4 min, A/B: 70/30 (v/v) gradient for 2 min., A/B: 70/30 (v/v) then A/B: 100/0 (v/v) gradient for 5 min. The column was equilibrated under the conditions given above for 10 minutes.

### Example 1

Preparation of Silica Monoliths Incorporating a Candida rugosa Lipase

In this example, the preparation of a silica monolith and the immobilization of a Candida rugosa lipase in the macropores of this monolith are illustrated.

1) First step: Synthesis of the silica monolith (MSi).

5.0 g of TEOS were added to 16.0 g of an aqueous solution of TTAB at 35% by weight, acidified beforehand with 7 g of a 37% concentrated hydrochloric acid solution. The mixture was left to hydrolyze with stirring for approximately 5 minutes until a single-phase hydrophilic medium (aqueous phase of the emulsion) was obtained. Next, 35.0 g of dodecane (oily phase of the emulsion) were added dropwise to this aqueous phase, with stirring. The mixture was transferred into a cylindrical Teflon® container (L=251.0 mm; r=9.65 mm) acting as a macroscopic mold, said container being itself contained in a steel chromatography column with a working length of 300 mm and an internal diameter of 19 mm, sold by the company Interchim (Montluçon, France). The emulsion was then left to condense in the form of a silica monolith for 1 week at ambient temperature. The silica monolith thus synthesized was then washed for 4 days by continuous flow circulation of a tetrahydrofuran/acetone (50/50:v/v) mixture at a rate of 0.1 ml/min in order to extract the oily phase of the monolith.

The resulting monolith had the following morphological characteristics:
porosity: 94%
density of the monolith: 0.07 g·cm⁻³
density of the silica backbone: 1.23 g·cm⁻³
specific surface area: 210 m²·g⁻¹ (BET) and 190 m²·g⁻¹ (BJH).

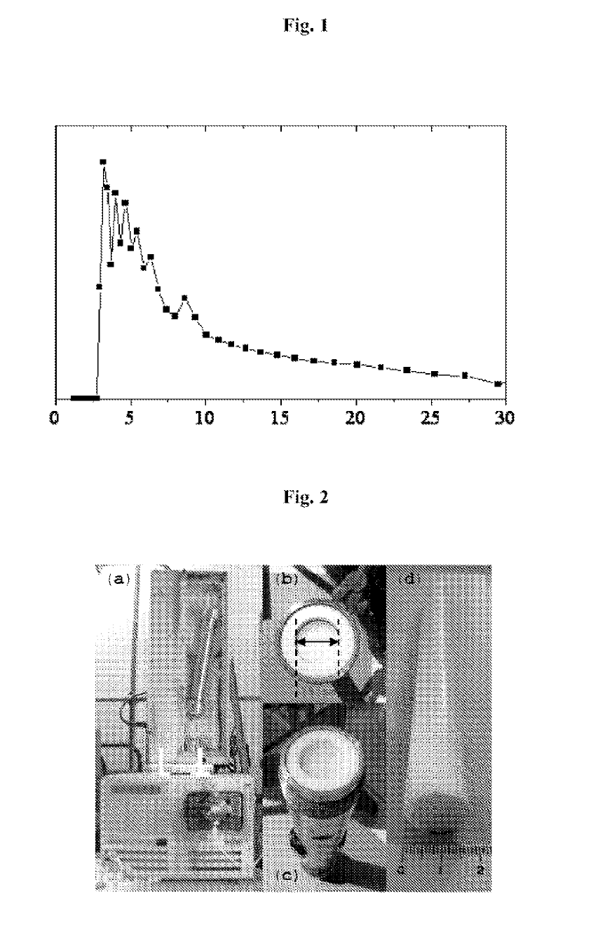

The results of the mercury intrusion measurements carried out on this monolith are given in appended FIG. 1, in which the differential pore volume (in arbitrary units) is as a function of the pore diameter (in nm).

It can be noted that the monolith obtained is free of micropores.

2) Second step: Functionalization of the silica monolith with a coupling agent of silane type

The silica monolith obtained above in the preceding step was then functionalized with Glymo.

To do this, 200 ml of a 0.05 M solution of Glymo in chloroform were passed through the chromatography column containing the monolith obtained above in the preceding step, in continuous flow and in a closed circuit, at a rate of 0.1 ml·min⁻¹ for 3 days.

The monolith was next washed with chloroform and then with acetone and, finally, with water in continuous flow at a rate of 0.5 ml·min⁻¹.

A silica monolith in which the surface of the macropores is functionalized with Glymo (MSi-Glymo) was thus obtained.

3) Second step: Immobilization of the lipase in the MSi-Glymo macropores

500 mg of unpurified Candida rugosa lipase (lCR) were dispersed in 200 ml of distilled water and mixed for one hour at ambient temperature until a solution was obtained. The immobilization of the lipase in the macropores of the MSi-Glymo monolith obtained above in the preceding step was then carried out by circulating the lipase solution, in a closed circuit and in ascendant flow, at a rate of 0.1 ml·min⁻¹ for 5 days at ambient temperature.

The chromatography column impregnated with the lipase solution was then left to stand for 1 month at 4° C. The chromatography column containing the monolith was then washed, in ascending continuous flow, with distilled water, in order to completely remove the lipases that had not been immobilized in the macropores of the monolith. Total removal of the lipases was monitored by the Bradford method, which is a colorimetric protein assay based on the change in absorbance (the measurement is carried out at 595 nm), which shows up through the changing the color of Coomassie blue after bonding (complexation) with the aromatic amino acids (tryptophan, tyrosine and phenylalanine) and the hydrophobic residues of the amino acids present in the protein(s) (M. M. Bradford, Anal. Biochem., 1976, 72, 248-254).

The chromatography column was then washed with heptane under continuous flow at a rate of 0.1 ml·min⁻¹.

A heterogeneous catalyst in accordance with the invention (MSi-Glymo-lRC): i.e. a cellular silica monolith comprising macropores and mesopores, free of micropores, and the macropores of which contain a lipase immobilized by means of Glymo, was obtained. In this monolith, the macropores had sizes ranging from 10 to 50 μm approximately.

### Example 2

Preparation of a Silica Monolith Incorporating a Thermomyces lanuginosus Lipase

In this example, the preparation of a silica monolith and the immobilization of a Thermomyces lanuginosus lipase in the macropores of this monolith are illustrated.

4 g of unpurified Thermomyces lanuginosus lipase (lTL) were dispersed in 200 ml of distilled water and mixed for one hour at ambient temperature until a solution was obtained. The immobilization of the lipase in the macropores of an MSi-Glymo monolith as obtained above at the end of step 2) of example 1 was then carried out by circulating the lipase solution, in a closed circuit and in ascending flow, at a rate of 0.1 ml·min⁻¹ for 5 days at ambient temperature.

The chromatography column impregnated with the lipase solution was then left to stand for 2 weeks at 4° C., and then washed with water until disappearance of the absorbance according to the Bradford method in order to completely remove the nonimmobilized lipases.

The chromatography column containing the monolith was then impregnated by circulation in a closed circuit of 200 ml in an aqueous 5% (weight/volume) glutaraldehyde solution, under ascending continuous flow at a rate of 0.1 ml·min⁻¹ for 3 days at ambient temperature.

The chromatography column containing the monolith was then again impregnated with a new solution of lTL lipase (4 g of lipase for 200 ml of distilled water) under the same conditions as previously.

The chromatography column was then washed with distilled water until disappearance of the absorbance according to the Bradford method in order to completely remove the nonimmobilized lipases, and then with heptane (0.1 ml·min⁻¹ in ascending continuous flow), for 3 days.

A heterogeneous catalyst in accordance with the invention (MSi-Glymo-lTL), i.e. a cellular silica monolith comprising macropores and mesopores, free of micropores, and the macropores of which contain a lipase immobilized by means of Glymo, was obtained. In this monolith, the macropores had sizes ranging from 10 to 50 μm approximately.

### Example 3

Esterification of Oleic Acid with 1-butanol, in the Presence of an Enzymatic Catalyst in Accordance with the Invention

In this example, a catalyzed reaction for esterification of oleic acid (1) with 1-butanol (2) was carried out according to the following reaction scheme:

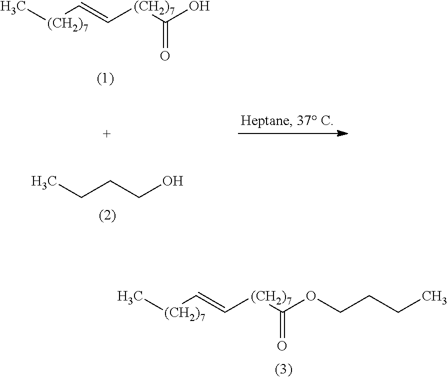

This reaction results in the formation of oleic acid butyl ester (3).

A reaction medium containing 23.0 mmol·l⁻¹ of oleic acid (1) and 46.0 mmol·l⁻¹ of 1-butanol (2) in heptane was prepared.

The reaction medium was passed, in ascending continuous flow, at 37° C., through the chromatography column provided with the MSi-Glymo-lCR catalyst as prepared above in example 1, at an initial rate of 0.05 ml·min⁻¹ for 20 days, then at a rate of 0.1 ml·min⁻¹ for 25 days and, finally, at a rate of 0.05 ml·min⁻¹ for 5 days. The formation of the ester (3) was monitored by HPLC.

The esterification reaction was thus carried out continuously for a total period of 50 days.

The appended FIG. 2 represents a photograph of the whole of the reaction device (FIG. 2a), of the solid catalyst in accordance with the invention after 60 days of continuous flow reaction: inside its mold and the chromatography column (2b and 2c), and of the solid catalyst after 50 days of continuous flow catalysis, after having removed it from its mold (2d). It is noted that, after 50 days of continuous flow use, integrity of the monolith is preserved.

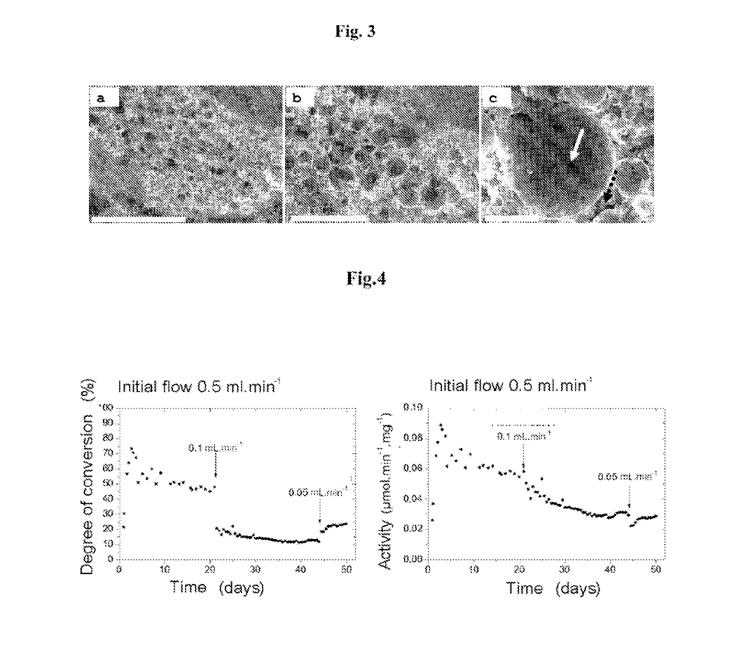

The appended FIG. 3 represents photographs taken by SEM of the catalyst in section after 50 days of continuous flow reaction, washing with distilled water and lyophilization (magnification×200: 3a; magnification×800: 3b and magnification×1500: 3c). It is also noted that the macroporous structure of the monolith is preserved. In FIGS. 3a and 3b, the interconnected macropores of the monolith can be seen. In FIG. 3c, the white arrow represents the internal cellular junction, while the dashed black arrow represents the external cellular junction.

The esterification results obtained are given in the appended FIG. 4. FIG. 4a gives the level of formation of oleic acid butyl ester (3), expressed as a percentage, as a function of the time in days. FIG. 4b gives the enzymatic activity of the catalyst (in μmol·min⁻¹·mg⁻¹) as a function of the time in days. The results given in this figure show that, after 50 days of reaction, the activity of the enzyme is still equal to 50% of the initial activity, which is unprecedented for an enzymatic catalyst used in continuous flow.

### Example 4

Transesterification of Linoleic Acid Triglycerides Contained in Crude Safflower Oil in the Presence of an Enzymatic Catalyst in Accordance with the Invention

In this example, the transesterification of the linoleic acid triglycerides (4) contained in crude safflower (Carthamus tinctorius) oil with ethanol (2) was carried out according to the following reaction scheme:

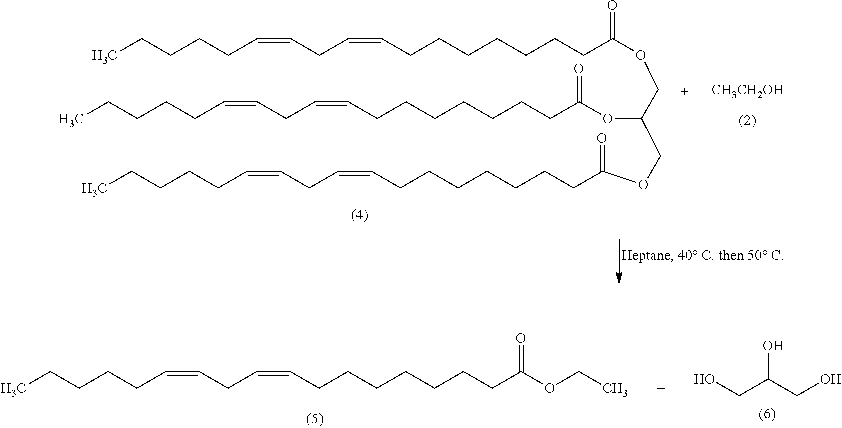

This reaction results in the formation of linoleic acid ethyl ester (5) and glycerol (6). Such a reaction is used for the production of biodiesels which are methyl or ethyl esters of plant oils.

A reaction medium containing 38% by weight of safflower oil, 12% by weight of ethanol and 50% by weight of heptane was prepared.

The reaction medium was passed, in ascending continuous flow, at 40° C. for the first ten days, then at 50° C. for the subsequent days, through the chromatography column provided with the MSi-Glymo-lTL catalyst as prepared above in example 2, at a rate of 0.05 ml·min⁻¹. The formation of the ester (5) was monitored by HPLC.

The esterification reaction was thus carried out continuously for a total period of 60 days.

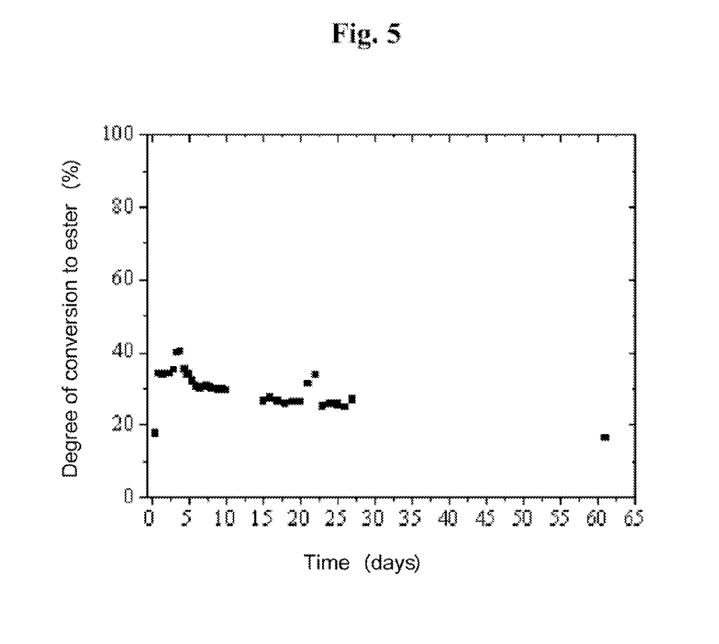

The results obtained are given in the appended FIG. 5 in which the degree of conversion to ester (5) expressed as a percentage is a function of the time in days.

These results show that the MSi-Glymo-lTL heterogeneous catalyst can be used in continuous flow for catalyzing the transesterification of safflower oil triglycerides with a degree of conversion which is still 20% after 60 days.

All the results given in these examples demonstrate that the heterogeneous enzymatic catalysts in accordance with the invention make it possible to catalyze various chemical reactions in continuous flow, with yields that are higher than the yields normally obtained in the prior art.

1. A heterogeneous enzymatic catalyst, in the form of a cellular monolith comprising:
a silica monolith, said monolith being free of micropores and having macropores having a mean size d_{A} of from 1 μm to 100 μm and mesopores having a mean size d_{E} of from 2 to 50 nm, said macropores being interconnected, and in which the internal surface of the macropores is functionalized with a coupling agent, chosen from silanes, to which an enzyme is attached by means of either one of a covalent or electrostatic bond.

2. The catalyst as claimed in claim 1, wherein the enzyme immobilized is an unpurified enzyme.

3. The catalyst as claimed in claim 1, wherein the macropores have a mean size d_{A} ranging from 10 to 100 μm.

4. The catalyst as claimed in claim 1, wherein said monolith has a specific surface area of from 200 to 1000 m²/g.

5. The catalyst as claimed in claim 1, wherein the coupling agent is chosen from silanes selected from the group consisting of γ-glycidoxypropyltrimethoxysilane; silylated ionic liquids and silanes of formula Si(OR²)₃R³ in which R² represents a C₁-C₂ alkyl group, and R³ represents a —(CH₂OH—CH₂OH)_{q}—CH₂OH or —(CH₂OH—CH₂OH)_{q}—CH₂CH₃ group in which q is an integer ranging from 1 to 10.

6. The catalyst as claimed in claim 5, wherein the coupling agent is γ-glycidoxypropyltrimethoxysilane.

7. The catalyst as claimed in claim 1, wherein the enzyme is selected from the group consisting of hydrolases, lyases, isomerases and oxidoreductases.

8. The catalyst as claimed in claim 7, wherein the enzyme is a hydrolase selected from the group consisting of esterases.

9. The catalyst as claimed in claim 8, wherein the enzyme is an esterase selected from the group consisting of carboxylic ester hydrolases, aminoacylases, amidases and nitrilases.

10. The catalyst as claimed in claim 9, wherein the enzyme is selected from the group consisting of Candida rugosa, Candida antartica, Aspergillus niger, Aspergillus oryzae, Thermomyces lanuginosus, Chromobacterium viscosum, Rhizomucor miehei, Pseudomonas fluorescens, Pseudomonas cepacia, Penicillium roqueforti, Penicillium expansum and Rhizopus arrhizus and lipases and wheatgerm lipases.

11. The catalyst as claimed in claim 1, wherein the amount of enzyme immobilized ranges from 1% to 40% by weight relative to the total weight of the catalyst.

12. A process for preparing a heterogeneous enzymatic catalyst as defined in claim 1, said process comprising the following steps:
1) a first step of preparing a cellular monolith consisting of a silica matrix, said monolith being free of micropores and comprising macropores having a mean size d_{A} of from 1 μm to 100 μm and mesopores having a mean size d_{E} of from 2 to 50 nm, said pores being interconnected, said first step comprising the following substeps:
1a) preparing an emulsion by introducing an oily phase into an aqueous surfactant solution,
1b) adding an aqueous solution of at least one silica oxide precursor to the surfactant solution, before or after preparation of the emulsion,
1c) introducing the reaction mixture into a mold,
1d) leaving the reaction mixture to stand in the mold until said silica precursor has condensed in the shape of said monolith,
1e) washing said monolith, in continuous flow, with an organic solvent;
2) a second step of functionalizing the internal surface of the macropores with a coupling agent selected from the group consisting of silanes, by impregnating the cellular monolith, in continuous flow, with a solution of the coupling agent in an organic solvent; and
3) a third step of immobilizing at least one enzyme on the coupling agent by means of a covalent bond, by impregnating the thus functionalized monolith, in continuous flow, with either one of an aqueous solution or an aqueous dispersion of at least one enzyme.

13. The process as claimed in claim 12, wherein the mold used in the first step is itself contained inside a device allowing continuous flow circulation of a liquid.

14. The process as claimed in claim 12, wherein the continuous flows mentioned in steps 2) and 3) of said process are ascending continuous flows.

15. The process as claimed in claim 12, wherein, during steps 2) and 3), the flow rate ranges from 0.02 to 0.1 ml/min.

16. The process as claimed in claim 12, wherein the immobilizing step 3) is repeated twice and in that the process also comprises, before carrying out step 3) for the second time, an additional step of impregnating the monolith, in continuous flow, with a solution of an aldehyde.

17. The process as claimed in claim 12, wherein the silica precursor(s) is (are) selected from the group consisting of tetramethoxyorthosilane, tetraethoxyorthosilane, dimethyldiethoxysilane, mixtures of dimethyldiethoxysilane with tetraethoxyorthosilane or tetramethoxyorthosilane, mixtures of tetramethoxyorthosilane or of tetraethoxyorthosilane with γ-glycidoxypropyltrimethoxysilane, and mixtures of dimethyldiethoxysilane or of γ-glycidoxypropyltrimethoxysilane with a silicate.

18. The process as claimed in claim 12, wherein the monolith functionalized with the coupling agent, as obtained at the end of the functionalizing step 2), is washed, under continuous flow, with an organic solvent.

19. A method for carrying out continuous flow heterogeneous-phase catalyzed chemical reactions, said method comprising the step of:
employing a heterogeneous enzymatic catalyst as defined in claim 1.

20. The method as claimed in claim 19, wherein the enzyme is a lipase, for catalyzing the hydrolysis of fatty acid triglycerides, esterification reactions between an acid and an alcohol, transesterification reactions between an ester and an alcohol, inter-esterification reactions between two esters or reactions for transfer of an acetyl group of an ester to an amine or to a thiol.

21. The method as claimed in claim 20, wherein said method is applied for catalyzing any one of:
the synthesis of butyl oleate;
the hydrolysis of glycerol-linoleic ester derivatives to result in soaps or detergents;
reactions for transesterification of fatty acid triglycerides with an alcohol, said reactions being involved in the synthesis of low-viscosity biodiesels.
